(19) United States
(12) Patent Application Publication
(10) Pub. No.: US 2015/0000676 A1
(43) Pub. Date: Jan. 1, 2015

(54) Surgical Drape and Attachment Mechanism

(71) Applicant: John A. Colona, Mount Pleasant, SC (US)

(72) Inventor: John A. Colona, Mount Pleasant, SC (US)

(21) Appl. No.: 14/312,314

(22) Filed: Jun. 23, 2014

Related U.S. Application Data
(60) Provisional application No. 61/841,465, filed on Jul. 1, 2013.

(51) Int. Cl. A61B 19/08

(52) U.S. Cl. CPC A61B 19/08
USPC 128/849

(57) ABSTRACT

The present invention provides a means to affix a surgical drape to an IV pole, operating table, and, or piece of other medical equipment. The present invention contemplates a surgical drape, with an attachment surface comprised of VELCRO™ backing, adhesive backing, magnetic backing, hooks, loops, a snatch, or another means of attachment. The present invention further contemplates a complimentary attachment surface that is affixed directly to a corresponding attachment surface on said drape, a device, an IV pole, operating table, and, or piece of other medical equipment.

### CROSS-REFERENCE TO RELATED APPLICATIONS

This application claims the benefit of provisional patent application No. 61/841,465, filed Jul. 1, 2013, by the present inventor.

### FEDERALLY SPONSORED RESEARCH

Not applicable.

### SEQUENCE LISTING

Not applicable.

### TECHNICAL FIELD

The present invention relates to an affixation device and complimentary surgical drape, and the method of attaching and securing such a surgical drape during a medical procedure.

### BACKGROUND OF INVENTION

During surgical procedures, a patient and the surrounding areas are covered with a sterile barrier known as a drape. The purpose of draping, as it is known in the art, is to minimize the passage of microorganisms between sterile and non-sterile areas. Surgical drapes may be disposable or non-disposable. In general, surgical drapes are resistant to blood, aqueous fluid, and abrasion, and are relatively lint-free. To be effective and to minimize the costs and risks associated with contamination, a surgical drape must be secured to the patient, operating table, IV pole or other medical devices, with a clamp, clip, tape or other means of attachment. The sterility of surgical drapes depends on the drape remaining dry and undisturbed following placement.

To drape a patient, a medical professional carries the folded drape to the operative site, places the drape in its proper position, and then unfolds the drape. Following placement of the drape, the drape should not be moved. The drape should be immediately secured.

Currently, in the art, medical professionals secure surgical drapes to objects using multiple methods of affixation, including clamps, clips and tape. The attachment mechanisms currently used in the art are cost, time and safety prohibitive. Currently used draping mechanisms fail to provide a consistent and secure barrier between sterile and non-sterile areas, thereby compromising the health and safety of patients and medical personnel.

In the operating room, at times, clamps, clips, tape, and other means of securing drapes to patients, IV poles and operating tables, are left unattended or unsecured by hospital staff There is a risk that unsecured clamps, clips, or other means of attachment may fall into a patient, onto the floor, or otherwise become lost during a procedure. Pursuant to most hospital policies, equipment counts are accomplished post-surgery. In the event of a miscount, an x-ray of the patient must be accomplished to verify the missing piece of equipment was not left inside the patient by mistake. This procedure increases the medical costs to the patient and, or hospital. It also increases the time spent in the operating room, which likewise increases medical costs.

The current method of securing surgical drapes can be dangerous to medical providers. Currently used attachment devices, like certain clamps, have sharp points. When passing these devices or working to secure these devices, a medical professional can be easily injured, and the sterility of the medical professional's glove, and by default the operating room, may be compromised.

Other clamping devices do not properly secure the drape. If a drape falls and touches a non-sterile area, moves, or adjusts in such a manner so as to compromise the sterility of the operating room, the drape must be discarded and the patient must be re-draped.

In the event operating room sterility or patient safety is compromised because of a surgical drape malfunction, the time and costs associated with a medical procedure may increase. Extended time in the operating room or in a hospital may lead to increased morbidity and mortality, which may, in turn, lead to potential litigation.

The current means of attaching a surgical drape places undue time and labor burdens on a surgical team, as the services of more than one medical professional are typically required to place and secure a surgical drape. In a fast-paced environment where staff members often carry multiple responsibilities, it may be difficult to locate two or more medical professionals who are available to assist with draping at the same time. Therefore, valuable time in the operating room may be lost where one member of the surgical team is forced to wait on another member to assist him in securing a surgical drape. This delay may also lead to increased labor and medical costs.

There is a need in the art for an improved affixation device and complimentary surgical drape that will allow medical personnel greater flexibility in positioning and securing a drape during a medical procedure. A device of the present invention will allow medical professionals to affix a surgical drape to an affixation device that attaches to an IV pole, operating table and, or other piece of medical equipment. Or, in the alternative, the present invention contemplates a self-affixing surgical drape or a surgical drape that directly affixes to an IV pole, operating table, and, or piece of other medical equipment.

### BRIEF SUMMARY OF THE INVENTION

The present invention provides an affixation device and complimentary surgical drape and a method of securing and stabilizing said surgical drape during a medical procedure. The present invention further provides an affixation device capable of securely anchoring itself to an IV pole, operating table and, or piece of other medical equipment. The affixation device may be fixed or capable of transition. The present invention also provides a means to secure and attach a surgical drape.

In an alternative embodiment, the present invention contemplates securing a surgical drape to an IV pole, operating table, and, or piece of other medical equipment without the use of an affixation device. In such an embodiment, an attachment surface is placed directly to an IV pole, operating table piece of other medical equipment and, or the drape itself.

### DRAWINGS

The present invention will become more fully understood from the detailed description given herein and the accompanying drawings which are given by way of illustration only and thus are not limitive of the present invention, and wherein:

FIG. 1 is a perspective view of an affixation device according to an embodiment of the present invention.

FIG. 2 is a side illustration of the device of FIG. 1.

FIG. 3 is a side illustration of the device of FIG. 1.

FIG. 4 is a side illustration of the device of FIG. 1 wherein the attachment surface is partially engaged.

FIG. 5 is a schematic illustration of an affixation device according to an embodiment of the present invention as it is secured to an object during a medical procedure.

FIG. 6 is a schematic illustration of the device of FIG. 1 and a surgical drape according to an embodiment of the present invention as the surgical drape and affixation device are positioned during a medical procedure.

FIG. 7 is a perspective illustration of a surgical drape according to an embodiment of the present invention as it is being secured to an attachment surface of object during a medical procedure.

FIG. 8 is a perspective illustration of a medical professional attaching a drape according to an embodiment of the present invention to an object.

FIG. 9 is a rear view of a surgical drape according to an embodiment of the present invention as it is attached to an object during a medical procedure.

FIG. 10 is a rear view of a surgical drape according to an embodiment of the present invention as it is attached to an object during a medical procedure.

FIG. 11 is a perspective illustration of a surgical drape according to an embodiment of the present invention.

FIG. 12 is a perspective illustration of a surgical drape according to an embodiment of the present invention.

FIG. 13 is a perspective illustration of an attachment surface of a surgical drape according to an embodiment of the present invention that provides for anti-slip belting.

FIG. 14 is a perspective illustration of an attachment surface of a surgical drape according to an embodiment of the present invention that provides for anti-slip belting.

FIG. 15 is a perspective illustration of a surgical drape and affixation device according to an embodiment of the present invention secured to an object during a medical procedure.

FIG. 16 is a perspective illustration of a surgical drape according to an embodiment of the present invention secured to an object during a medical procedure.

FIG. 17 is a rear view of a surgical drape according to an embodiment of the present invention secured to an object during a medical procedure.

REFERENCE NUMERALS

| | |
|---|---|
| 10 | affixation device |
| 12 | finger grip |
| 14 | clamping member |
| 16 | pivot point |
| 18 | spring |
| 20 | housing |
| 22 | outer surface |
| 24 | inner surface |
| 26 | clamping end |
| 28 | handling end |
| 30 | attachment surface |
| 32 | medical professional |
| 34 | IV pole |
| 36 | surgical drape |
| 38 | attachment surface of surgical drape |
| 40 | sterile field |
| 42 | interior surface |
| 44 | patient |
| 46 | anti-slip belting |

### DETAILED DESCRIPTION OF THE INVENTION

The embodiments disclosed herein are discussed in the context of a surgical drape and complimentary affixation device because of the applicability and usefulness in such a field. More specifically, the surgical drape and complimentary affixation device of the present invention may be used to create a barrier between sterile and non-sterile areas in an operating room setting wherein said drape is easily and securely anchored to an IV pole, operating table or other medical device.

### First Embodiment

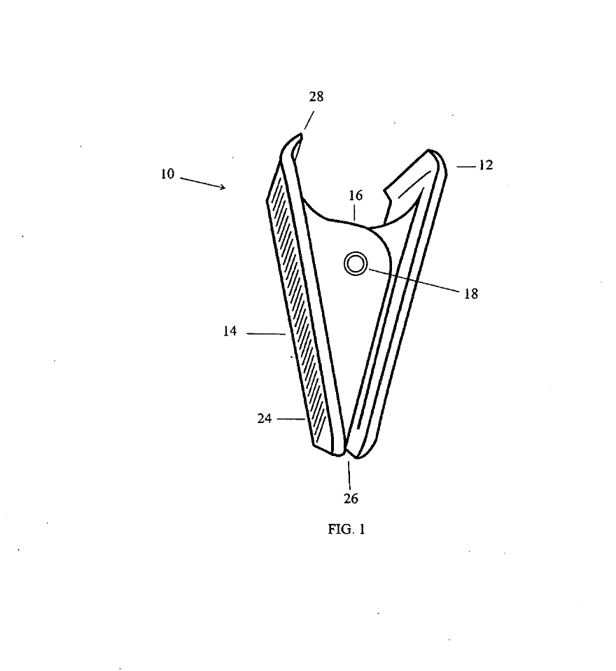

### FIGS. 1, 2, 3, 4, 5 and 6

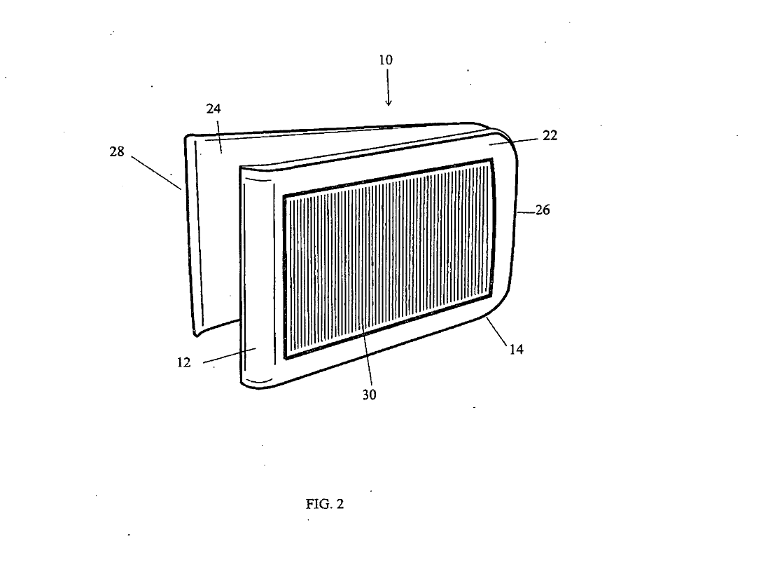

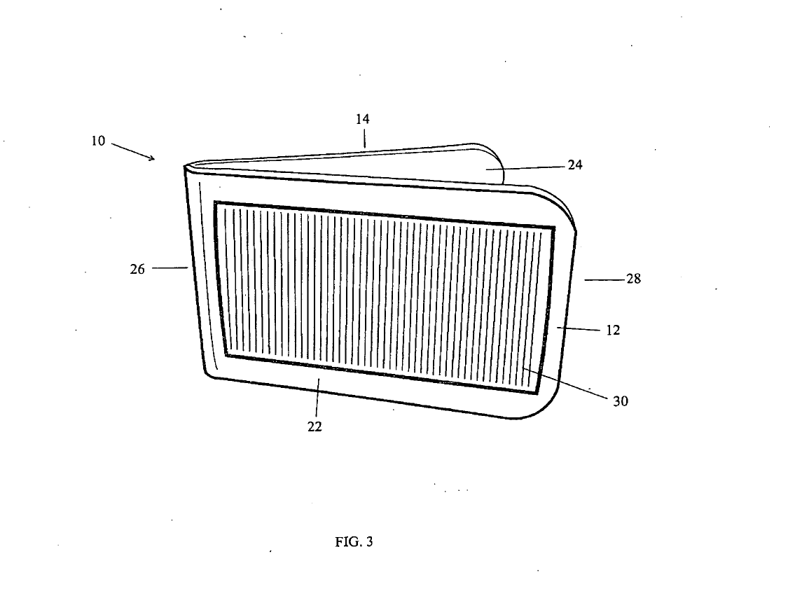

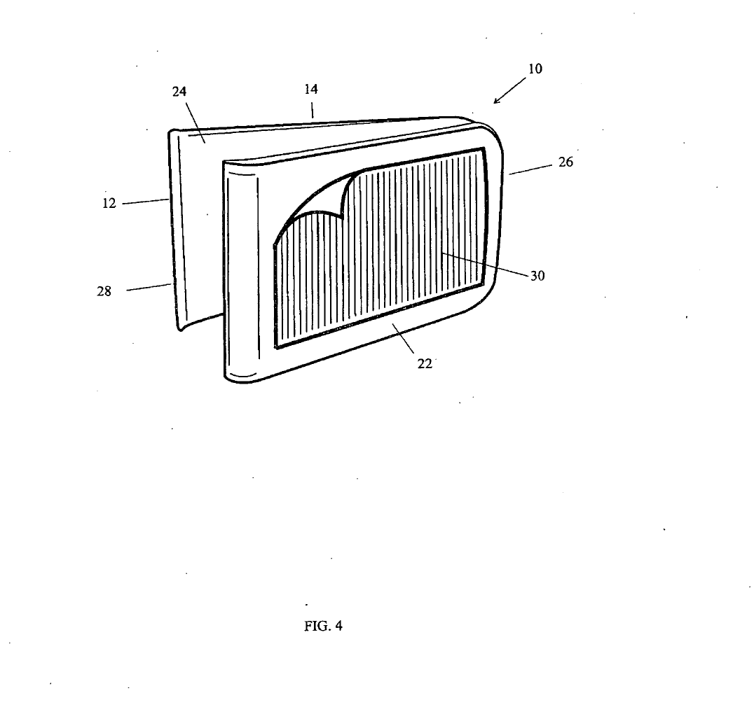

For a first embodiment, FIG. 1 (perspective illustration), FIG. 2 (side view), FIG. 3 (side view) and FIG. 4 (side view, attachment surface highlighted) generally depict an affixation device 10. The affixation device 10 is used to secure a surgical drape in the context of a medical operation wherein the surgical drape creates a barrier between sterile and non-sterile surfaces.

The affixation device 10 is sized to accommodate, support and secure a surgical drape. The affixation device 10 is designed to attach to an IV pole, operating table or other piece of medical equipment. In most embodiments, the affixation device 10 will generally range from about 50 to 800 millimeters (mm) in width. The affixation device 10 will generally range from 25 to 250 millimeters (mm) in height.

In the present embodiment, the width of the affixation device 10 does not taper. However, in alternative embodiments, it is contemplated that the width of an affixation device of the present invention may taper so that the width of the clamping member becomes gradually narrower.

Referring to FIG. 1, the affixation device 10 comprises two opposed clamping members 14 that are hinged together at a pivot point 16 by a spring 18 encased on a housing 20, that is located on the inner surface 24 of the affixation device 10. The pivot point 16 is a joint that allows for the two opposing clamping members 14 to be coupled together so that one clamping member can move relative to the other. The affixation device 10 further comprises a finger grip 12 on the outer surface 22 of each clamping member 14. The finger grip 12 is located on the handling end 28 of the affixation device 10. As will be more fully explained herein, the above parts work in tandem to facilitate the clamping process.

In an embodiment, in an unengaged state, the affixation device 10 comprises the shape of a “v” shape, wherein the clamping members 14 of the affixation device 10 transition to a point from the handling end 28 of the affixation device 10 to the clamping end 26 of the affixation device 10.

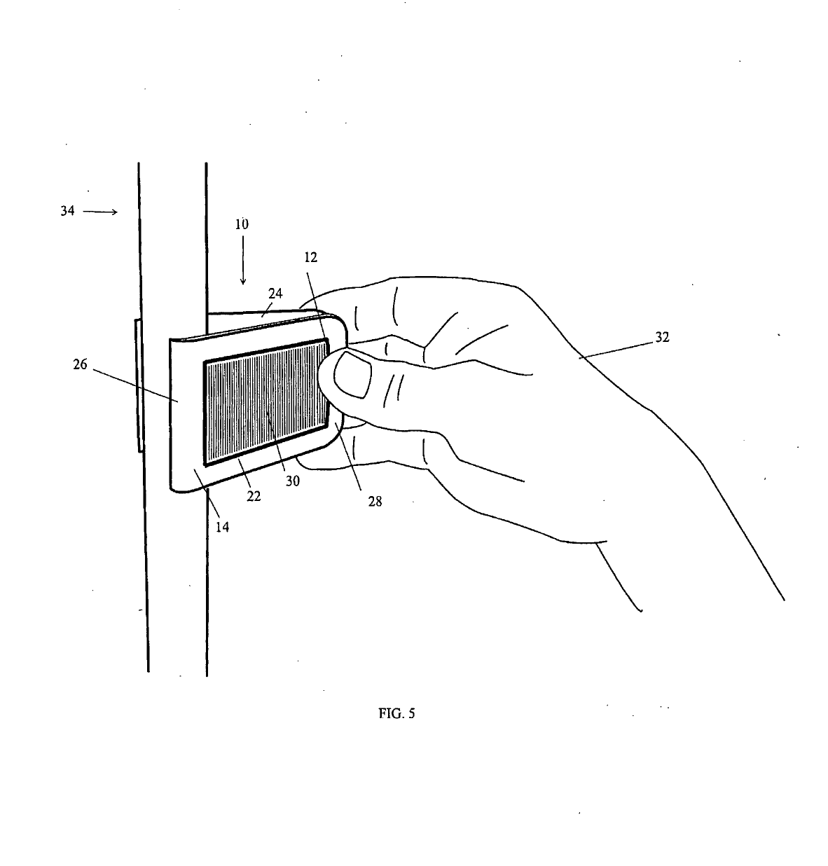

However, in an alternate embodiment, the overall shape of the affixation device may be of any shape that can attach to an IV pole, operating table or other piece of medical equipment and accommodate (see FIG. 5), support and secure a surgical drape. For example, it is contemplated that the affixation device may have a rectangular, cylindrical, bullet, cuboidal or polygonal shape. Similarly, the clamping end 26 of the affixation device may comprise any shape that will allow for attachment to an IV pole, operating table or other piece of medical equipment. Non-limiting examples include a rounded, beveled, rectangular, or polygonal clamping end.

In one aspect, the outer corners of the affixation device 10 are beveled or rounded so that it can accommodate an IV pole, operating table or other piece of medical equipment while maintaining contact with a surgical drape without snagging the drape or jabbing medical professionals on sharp or pointed corners during the attachment process. In other embodiments, the corners of the affixation device can be of any shape that will accommodate a surgical drape and, or allow for the attachment to an IV pole, operating table or other piece of medical equipment.

Referring to FIGS. 2 and 3, in an embodiment, the clamping members 14 of the affixation device 10 are planar or curved. Each clamping member 14 has an outer surface 22 and an inner surface 24. In an embodiment of the present invention, the overall shape of the clamping member 14 is rectangular. However, it is contemplated that in alternative embodiments the clamping member may comprise any shape that will allow the affixation device to attach to an IV pole, operating table or other medical device (see FIG. 5) and support the weight of and securely anchor a surgical drape. Non-limiting examples of the overall shape of an affixation device include circular, square, bullet and triangular shapes.

The components of a device of the present invention can be fabricated from various materials to allow such components to operate according to their intended function. For example, the components of the body of the affixation device can be fabricated from any metal, polymeric material or ceramic material.

Non-limiting examples of a metal include stainless steel, cobalt-chrome or titanium alloys. Non-limiting examples of plastics include a blend of polycaprolactone and polyglycolide, a blend of polyactide and polyglycolide, pure polydioxanone, poly (ethylene oxide), poly (butylene terephthalate), polyorthoester, or polyhydroxybutyrate. In certain embodiments, the components of the device are fabricated from biodegradable materials such as polycaprolactone, poly(L-lactide), polyglycol, poly(D,L-lactide), poly(D,L-lactide-co-glycol), poly(D,L-lactide-cocaptrolactone), polydioxanone, copolyoxalates and polycarbonates, such as, for example, polyglycol-co-trimethylenecarbonate and poly(glutamine-co-leucine).

Referring to FIG. 4, in an embodiment, the outer surface 22 of at least one clamping member 14 comprises an attachment surface 30. The attachment surface 30 is designed to complement and accept a surgical drape of the present invention, hereinafter described. In the present embodiment, the attachment surface 30 will cover a large portion of the surface area of a clamping member 14. In general, the attachment surface 30 will range from about 40 to 750 millimeters (mm) in width. The affixation device will generally range from 15 to 200 millimeters (mm) in height. However, in alternative embodiments, it is contemplated that the width and height the attachment surface may be of any size or surface area suitable to carry out its intended function.

The attachment surface 30 can be fabricated from various materials that will accommodate, attach to and adequately secure a complimentary surgical drape of the present invention, hereinafter described. For example, the attachment surface may be fabricated from VELCRO™ backing, adhesive backing, magnetic backing, hooks, loops or a snatch.

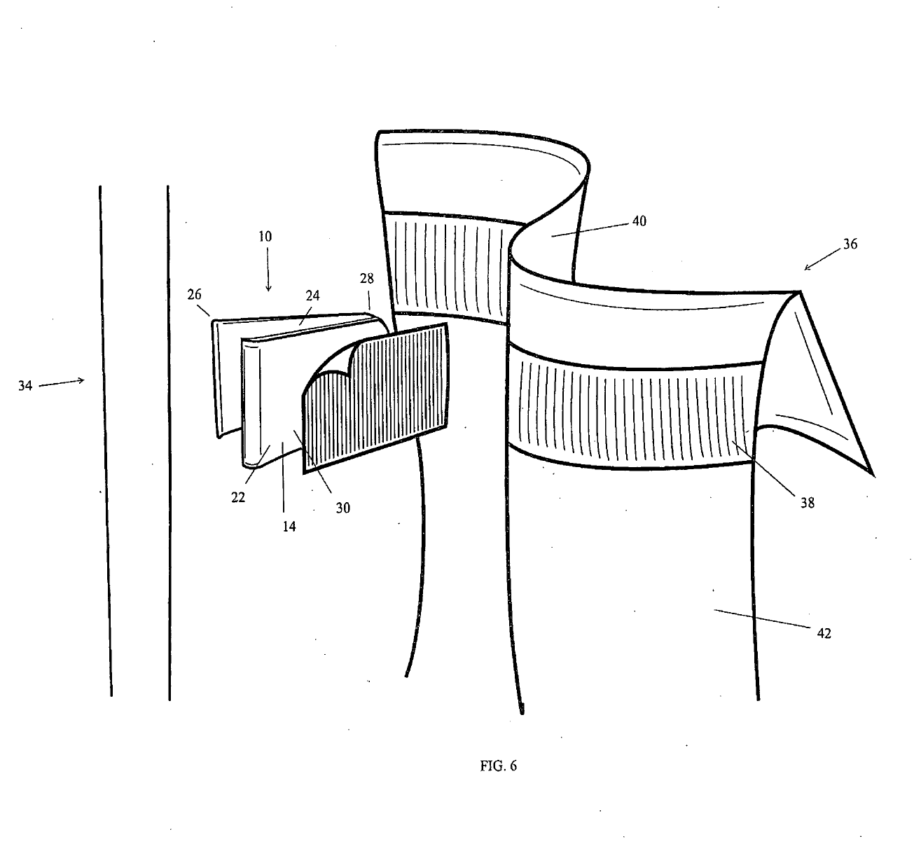

FIG. 6 generally depicts a surgical drape 36. The surgical drape is used in the context of a medical operation wherein the purpose of the surgical drape creates a barrier between sterile and non-sterile surfaces. In accordance with an embodiment of the present invention, a surgical drape 36 comprises an exterior surface adjacent to a sterile field 40 for performing a surgical procedure, and an interior surface 42 for receiving non-sterile surfaces. The drape further includes an attachment surface 38 located on the interior surface 42 of the surgical drape 36.

In an embodiment, the attachment surface of the surgical drape 38 is located at or near the top portion of the surgical drape 10, and extends the length and runs along the horizontal axis (not shown) of the surgical drape 38 and comprises an overall rectangular shape. In general, the attachment surface of the surgical drape 38 will range from about 40 to 750 millimeters (mm) in height and will run the length of the surgical drape. However, it is contemplated that the attachment surface of the surgical drape may be of any dimension, shape or location that will allow the surgical drape of the present invention to attach to an affixation device, as described above, of the present invention.

A surgical drape 38 of the present invention may be comprised of any barrier quality material. Said material may be woven or non-woven. Non-limiting examples of appropriate woven materials include cotton and linen. Non-limiting examples of appropriate non-woven materials include nylon, rayon and polyester.

The attachment surface of the surgical drape 38 can be fabricated from various materials that will allow, accommodate, attach to and adequately secure an affixation device of the present invention, as described above. For example, the attachment surface may be fabricated from VELCRO™ backing, adhesive backing, magnetic backing, hooks, loops or snatch.

### Alternative Embodiment

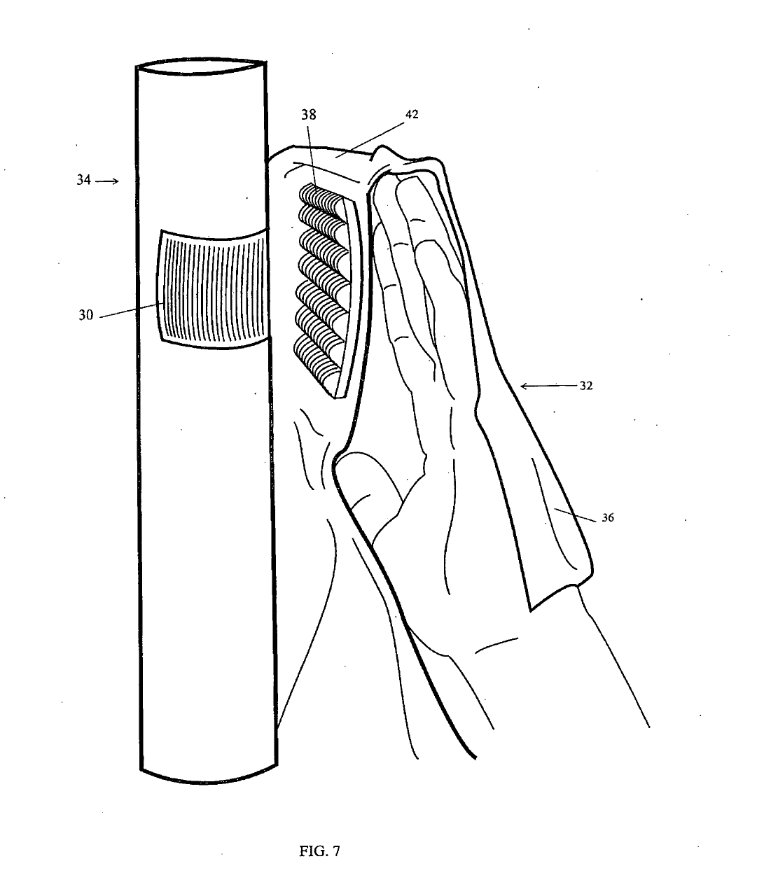

### FIGS. 7, 9, 10, 11, 12, 13 and 14

In an alternative embodiment, an affixation device, as described in the First Embodiment herein, is not required to secure a surgical drape during a medical procedure. FIGS. 7, 9, 10, 11, 12, 13 and 14 generally depict an attachment surface of the present invention that may be located on an object, like an IV pole 34, operating table (not shown), other piece of medical equipment (not shown), or on a surgical drape itself 36. In an alternative embodiment, a surgical drape 36 is secured directly to an attachment surface during the course of a medical operation to create a barrier between sterile and non-sterile surfaces.

Referring to FIG. 7, in an embodiment, the attachment surface 30 will generally range from 15 to 200 millimeters (mm) in height and 50 to 800 millimeters (mm) in width. However, in alternative embodiments, it is contemplated that the width and height the attachment surface may be of any size or surface area suitable to carry out its intended function. In addition, it is contemplated that multiple attachment surfaces may be utilized.

In an embodiment, the attachment surface 30 generally comprises an overall rectangular shape. However, in alternative embodiments, it is contemplated that the overall shape of the attachment surface may be of any shape suitable to carry out its intended function. Non-limiting examples of such shape include, square and circular shaped attachment surfaces.

The attachment surface 30 can be fabricated from various materials that will accommodate, attach to and adequately secure a complimentary surgical drape of the present invention. Non-limiting examples of the materials that the attachment surface may be fabricated from include VELCRO™ backing, adhesive backing, magnetic backing, hooks, loops or a snatch.

In an embodiment, FIGS. 9, 10, 11 and 12 generally depict a surgical drape 36, wherein the attachment surface is affixed directly to the surgical drape 36 such that the surgical drape is capable of self-affixing or self-adhering. The attachment surface of a surgical drape 38 is generally located at or near the top portion of the surgical drape 36. The attachment surface of a surgical drape 38 of the surgical drape 36 is generally located on the interior surface 42 of the surgical drape that is intended for receiving non-sterile surfaces. The attachment surface of a surgical drape 38 generally extends the length and runs along the horizontal axis (not shown) of the surgical drape 36 and comprises an overall rectangular shape. In an embodiment and as set forth in FIG. 9, it is contemplated that a surgical drape of the present invention may have two (2) unconnected attachment surfaces of a surgical drape 38 that are each of a generally rectangular shape. Such attachment surfaces of a surgical drape 38 may be fabricated from the same material, wherein such material allows for the surgical drape 36 to self-affix or self-adhere. Non-limiting examples of the materials that the attachment surface may be fabricated from include VELCRO™ backing, adhesive backing, magnetic backing, books, loops or a snatch.

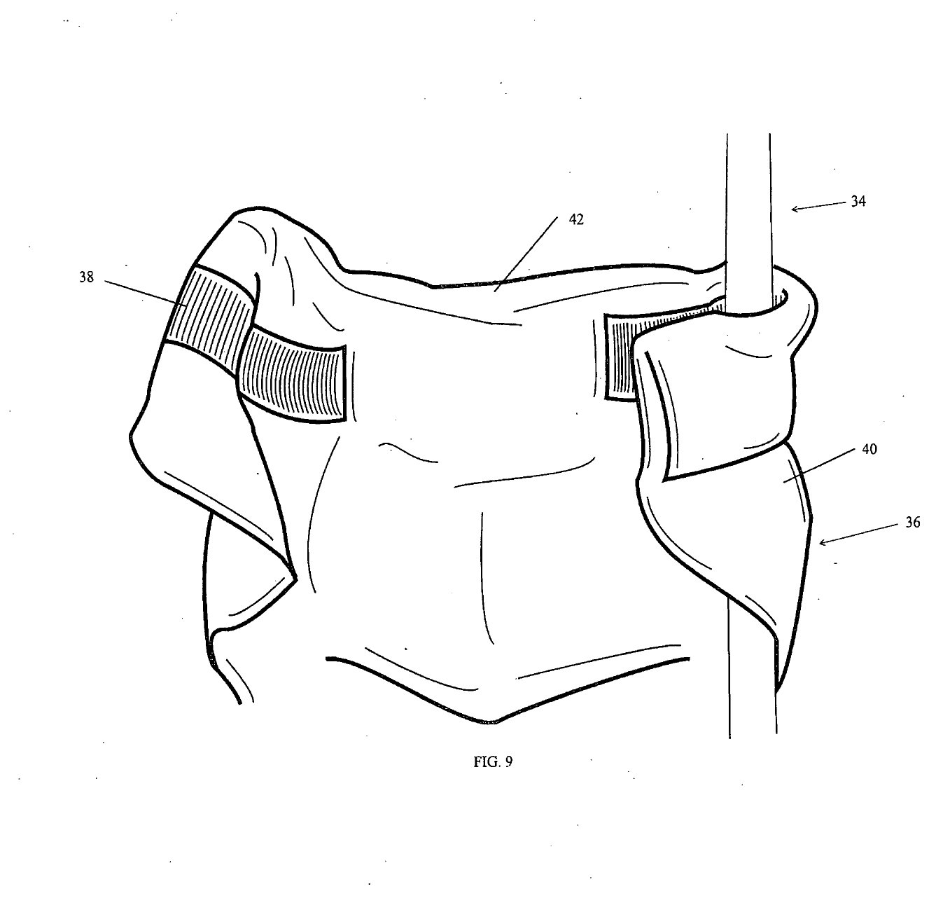

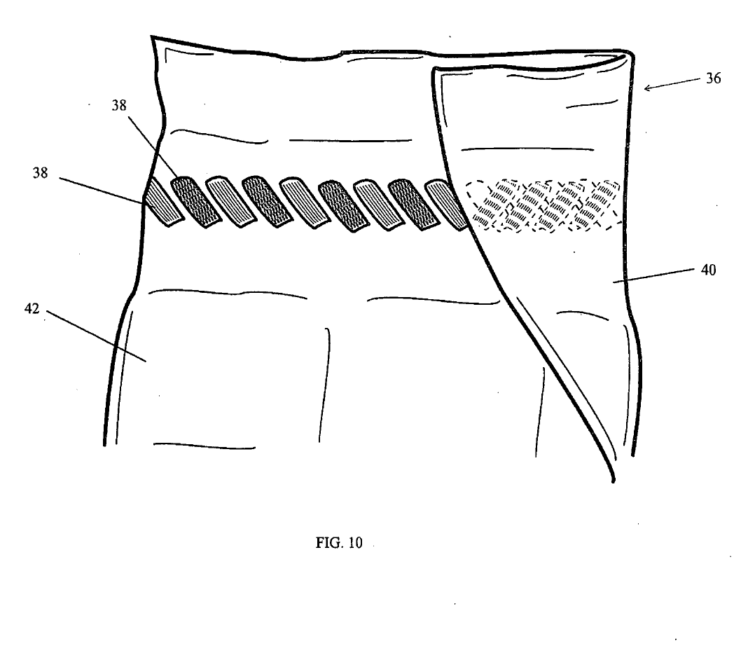

In an embodiment and as set forth in FIGS. 10 and 11; it is contemplated that a surgical drape of the present invention may have a plurality of unconnected attachment surfaces of a surgical drape 38 that are each of a generally rectangular shape. Such attachment surfaces of a surgical drape 38 may be fabricated from materials, wherein such material allows for the surgical drape 36 to self-affix or self-adhere. Non-limiting examples of the materials that the attachment surface may be fabricated from include VELCRO™ backing, adhesive backing, magnetic backing, hooks, loops or a snatch. It is further contemplated that the material that comprises the attachment surfaces of a surgical drape 38 may alternate or be composed of alternating complimentary materials.

Referring to FIGS. 10 and 11, the attachment surfaces of a surgical drape 38 are angled such that when the surgical drape 36 of the present embodiment is folded on itself, the attachment surfaces of a surgical drape 38 generally form an “x” shape. However, it is contemplated that such attachment surfaces may connect and, or attach so as to form any shape that will allow the surgical drape of the present embodiment to securely self-affix or self-attach.

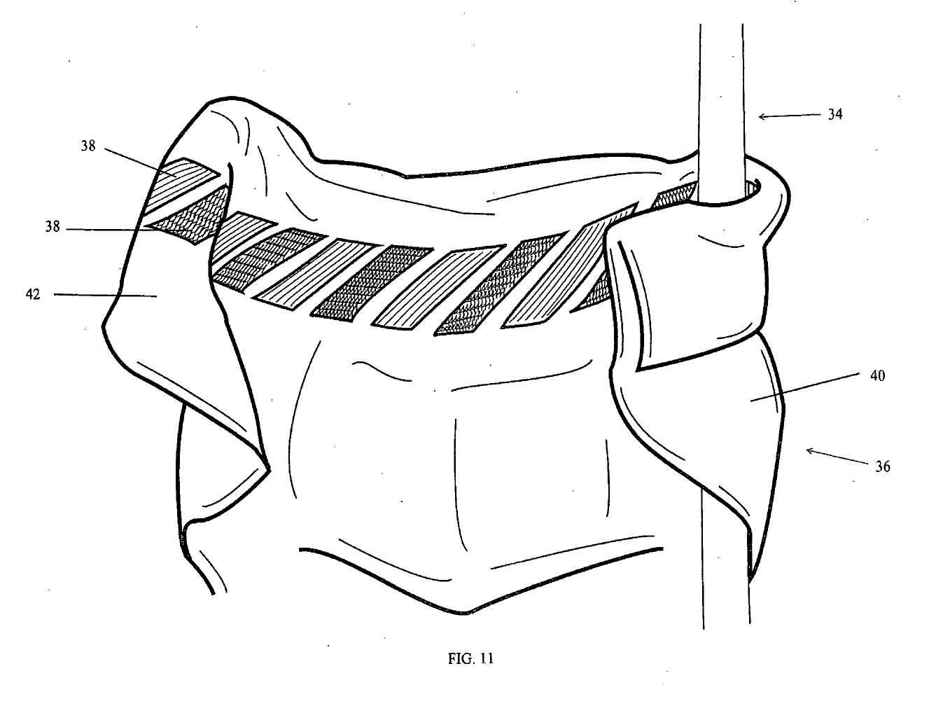

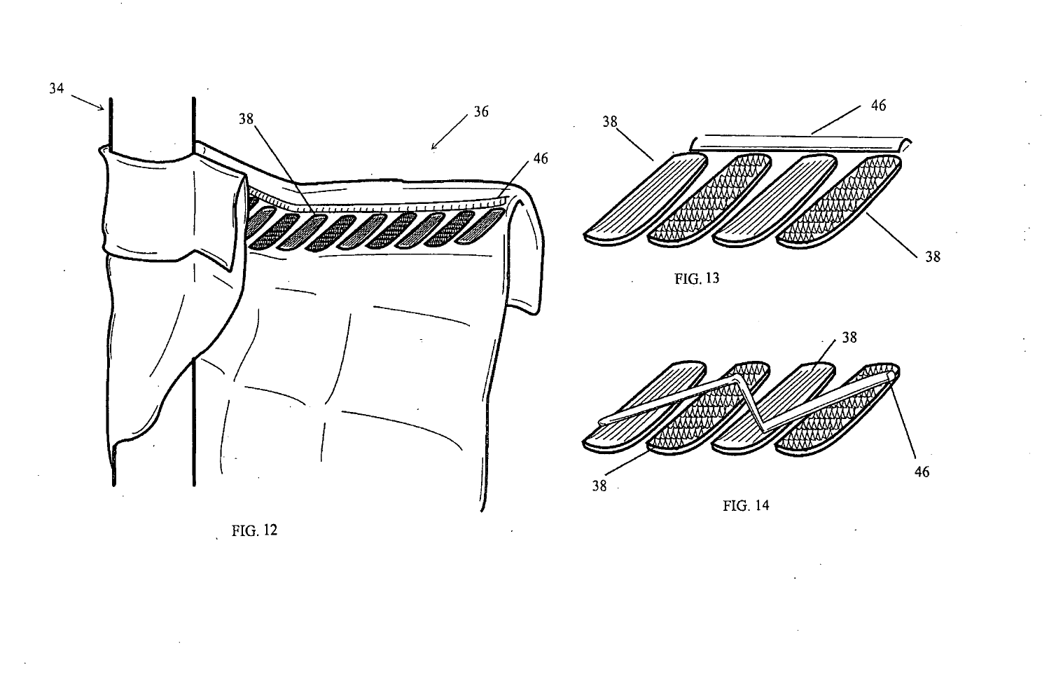

In an embodiment and as set forth in FIG. 12, it is further contemplated that a surgical drape 36 of the present invention may comprise an anti-slip belting 46 wherein such anti-slip belting 46 may be generally located on, at or near the attachment surface of a surgical drape 38.

The anti-slip belting 46 may be fabricated from various materials with adhesive and, or non-slip properties that will further assist a surgical drape of the present intention from slipping or sliding once attached or otherwise secured to an IV pole, operating table or other piece of medical equipment. Non-limiting examples of materials suitable for an anti-slip belting include Polyisoprene, Polychloroprene, Polybutadiene, Poly(styrene-butadiene-styrene), Polyisobutylene and Silicone.

In an embodiment, it is contemplated that the anti-slip belting 46 generally comprises an overall rectangular shape that extends along the horizontal axis (not shown) of the surgical drape 36. The anti-slip belting 46 will generally range from 5 to 75 millimeters (mm) in width and 50 to 1500 millimeters (mm) in length. However, in alternative embodiments, it is contemplated that the overall shape and size of the anti-slip belting may be of any shape suitable to carry out its intended function. In addition, it is contemplated that multiple runs of anti-slip belting may be utilized.

In an embodiment and as set forth in FIG. 13 it is contemplated that the anti-slip belting 46 runs the horizontal axis (not shown) of a surgical drape 36 of the present invention and is generally located immediately above an attachment surface of a surgical drape 38, such that the attachment surface of a surgical drape 38 and the anti-slip belting 46 are contiguous. However, in alternative embodiments, it is contemplated that the anti-slip belting may be located anywhere at or near the attachment surface of a surgical drape 38 suitable to carry out its intended function

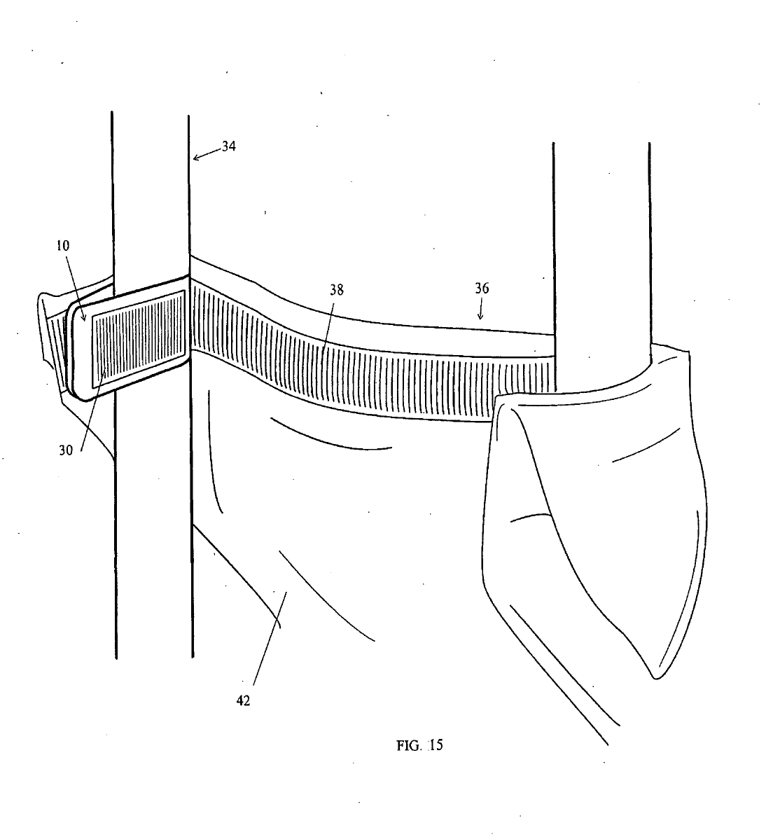

In an embodiment and as set forth in FIG. 14 it is contemplated that the anti-slip belting 46 is located directly on the attachment surface of a surgical drape 38, wherein the anti-slip belting 46 forms a “zig-zag” pattern on the attachment surface of a surgical drape 38. However, in alternative embodiments, it is contemplated that the anti-slip belting may be located anywhere on the attachment surface of the surgical drape 38 and form any pattern or be any configuration suitable to carry out its intended function

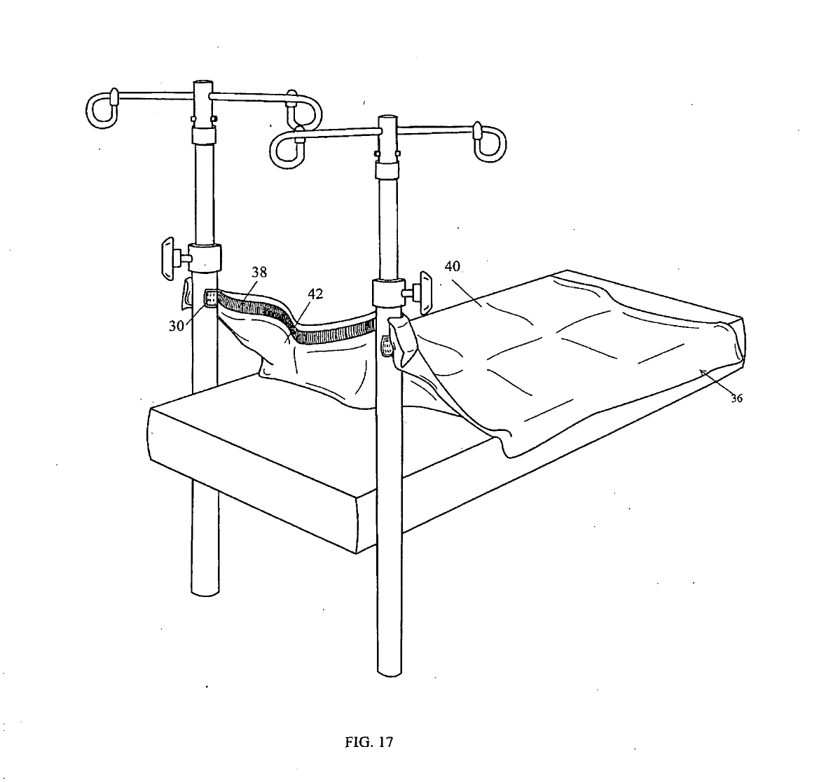

### Operation—FIGS. 5, 6, 8, 9, 10, 11, 15, 16 and 17

The manner of using a device of the present invention is similar to that for surgical drapes and affixation devices, like clamps, currently known in the art.

Referring to FIG. 5, an affixation device 10 of the present invention is attached to an IV pole 34, operating table (not shown) or other piece of medical equipment (not shown). As shown in FIG. 5, the affixation device may be compelled to an open position by applying pressure to the finger grip 12, located on the clamping member 14 of the affixation device 10. When the clamping members 14 move toward one another, the clamping end 26 of the affixation device 10 is forced open and can engage an IV pole 34 or other device. The clamping end 26 of the affixation device will close and tighten when pressure, as described above, is removed.

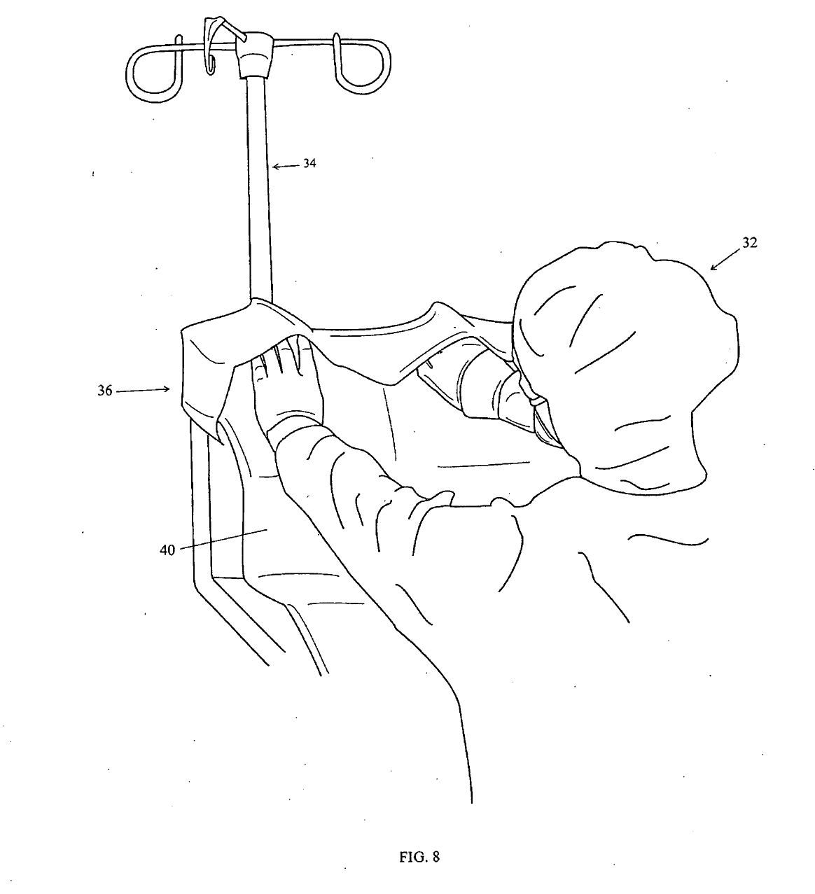

Referring to FIGS. 6 and 8, once an affixation device 10 of the present invention is secured to an IV pole 34, operating table (not shown) or other piece of medical equipment (not shown), the attachment surface 30 of the affixation device 10 can be engaged. The attachment surface 30 of the affixation device 10 is engaged wherein the attachment mechanism, non-limiting examples include VELCRO™ backing, adhesive backing, magnetic backing, hooks, loops or a snatch, is exposed.

Once properly positioned, the interior surface 42 of a surgical drape 36 of the present invention can be attached to an affixation device 10 of the present invention by lining the attachment surface of the surgical drape 38 with the attachment surface 30 of the affixation device 10 and applying manual force. It is anticipated that a medical professional 32 will apply said force.

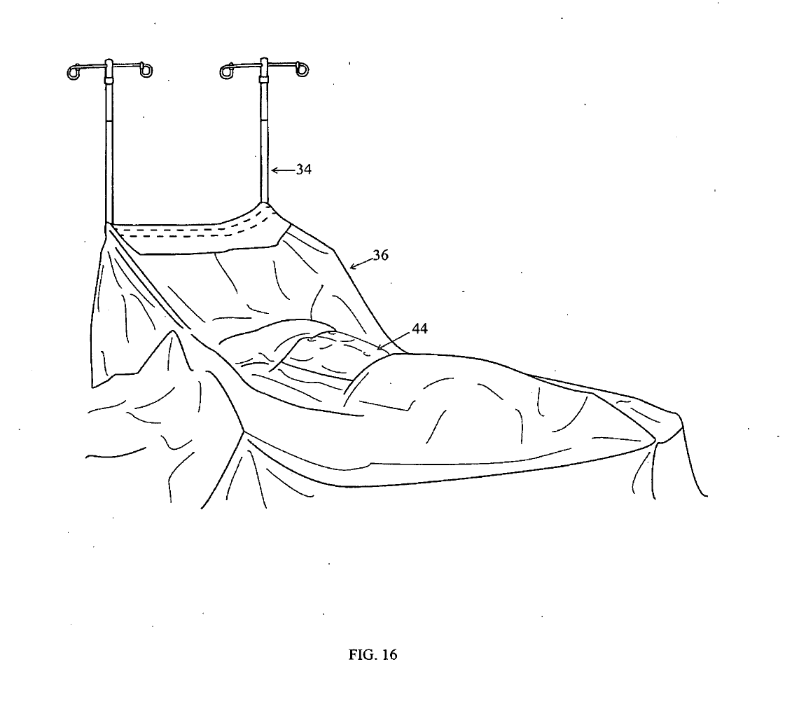

Referring to FIGS. 15, 16 and 17, an affixation device 10 and surgical drape 36 of the present invention offer complimentary surfaces that are designed to accept one another. Once the attachment surface 30 of the affixation device 10 is secured to the attachment surface of the drape 38, a surgical drape 36 of the present invention is designed to cover a patient 44 and provide a barrier between sterile and non-sterile surfaces areas the in operating room.

### Alternative Embodiment

### FIGS. 7, 9, 10, 11

Referring to FIGS. 7, 9, 10, and 11 in an alternative embodiment, a surgical drape 36 of the present invention may be secured to an IV pole 34, operating table (not shown) or other piece of medical equipment (not shown), without the use of an affixation device, as described herein in the forgoing embodiment.

Referring to FIG. 7, in an alternative embodiment, an attachment surface 30 is secured directly to IV pole 34, operating table (not shown) or other piece of medical equipment (not shown). The attachment surface 30 is engaged wherein the attachment surface 30 engages, connects, attached to or is secured by the attachment surface of the surgical drape 38, located on the interior surface 42 of a surgical drape 36 of the present invention. It is contemplated that the attachment surface 30 will be comprised of or will be fabricated from materials that are complimentary to the attachment surface of the surgical drape 38. Non-limiting examples of the materials that the aforementioned attachment surfaces can be comprised of or fabricated from include VELCRO™ backing, adhesive backing, magnetic backing, hooks, loops or a snatch, is exposed.

Referring to FIGS. 9, 10, and 11 in an alternative embodiment, it is contemplated that a surgical drape of the present invention will self-affix or self-adhere. Referring to FIGS. 9 and 11, a surgical drape 36 of the present invention is positioned such that the surgical drape 36 may be secured to an IV pole 34, operating table (not shown) or other piece of medical equipment (not shown) wherein a medical provider wraps or places the engages the attachment surface of the surgical drape 38, as located on the interior surface 42 of the surgical drape 36, by folding, wrapping or placing the attachment surface of the surgical drape 38, as located on the interior surface 42 of the surgical drape, on a complimentary attachment surface as located on the surgical drape 38.

It is contemplated that the attachment surface of the surgical drape 38 will be comprised of or will be fabricated from materials that are complimentary to the attachment surface of the surgical drape 38. Non-limiting examples of the materials that the aforementioned attachment surfaces can be comprised of or fabricated from include VELCRO™ backing, adhesive backing, magnetic backing, hooks, loops or a snatch, is exposed.

What is claimed is:

1. A surgical drape comprising:
an exterior surface adjacent to a sterile field for performing a surgical procedure, and an interior surface for receiving non-sterile surfaces, and
an attachment surface.

2. The surgical drape of claim 1, wherein said attachment surface is designed to accept, affix to, attach to, anchor, or secure said surgical drape to a complimentary attachment surface.

3. The surgical drape of claim 1, wherein said surgical drape comprises a plurality of attachment surfaces.

4. The surgical drape of claim 1, wherein said attachment surface comprises VELCRO™ backing, adhesive backing, magnetic backing, hooks, loops or a snatch.

5. The surgical drape of claim 1, wherein said surgical drape comprises anti-slip belting.

6. A device comprising:
a member having an attachment surface.

7. The device of claim 6, wherein said attachment surface is designed to accept, affix to, attach to, anchor, or secure said device to a complimentary attachment surface.

8. The device of claim 6, wherein said attachment surface comprises VELCRO™ backing, adhesive backing, magnetic backing, hooks, loops or a snatch.

9. A method for securing a surgical drape to a an IV pole, operating table or other piece of medical equipment, comprising:
positioning a surgical drape such that an attachment surface of said surgical drape accepts, affixes to, attaches to, anchors, or secures said attachment surface of said surgical drape to a complimentary attachment surface.
